(19) United States
(12) Patent Application Publication
(10) Pub. No.: US 2026/0224118 A1
(43) Pub. Date: Aug. 6, 2026

(54) METHOD AND SYSTEM FOR CALIBRATION-FREE ESTIMATION OF MEAN ARTERIAL PRESSURE FROM A SINGLE-LEAD ELECTROCARDIOGRAM

(71) Applicants: Monika Simjanoska Misheva, Skopje (MK); Kostadin Mishev, Skopje (MK)

(72) Inventors: Monika Simjanoska Misheva, Skopje (MK); Kostadin Mishev, Skopje (MK)

(21) Appl. No.: 19/542,820

(22) Filed: Feb. 18, 2026

(51) Int. Cl. A61B 5/021; A61B 5/352; A61B 5/355; G16H 50/30

(52) U.S. Cl. CPC A61B 5/021; A61B 5/352; A61B 5/355; G16H 50/30

(57) ABSTRACT

A method and system for the continuous, non-invasive estimation of mean arterial pressure (MAP) from a single-lead ECG signal. The system utilizes a hardware-deterministic pipeline comprising a signal acquisition stage, a filtering stage, and a landmark detection module. A hardware-accelerated fiducial mixture model (FMM) optimizer refines cardiac landmarks into a plurality of morphological parameters using a CORDIC processing unit. A thirty-element feature vector is extracted and stored in parameter-specific registers, including 18-bit phase registers and 10-bit timing interval registers. A deterministic decision-tree classifier processes the feature vector against pre-quantized thresholds to generate a MAP estimate with zero timing variance. The final estimate is delivered via an AXI-compatible output interface, enabling real-time monitoring within an integrated circuit architecture.

### CROSS-REFERENCE TO RELATED APPLICATIONS

This application is a completely new and original submission. The present invention does not claim priority to, and is not a continuation or divisional of, any prior application.

While foundational research regarding the general use of ECG signals in blood pressure estimation was previously described by the present inventor in WIPO Publication No. WO 2024/057067, that earlier methodology is fundamentally distinct from the present invention. The current submission introduces an entirely new hardware-deterministic architecture, a multi-domain morphological analysis engine utilizing a Fiducial Mixture Model (FMM), and specific register-transfer level (RTL) implementations, including parameter-specific 18-bit and 10-bit registers, which were neither disclosed nor enabled in any prior work.

### BACKGROUND OF THE INVENTION

The present invention relates generally to the fields of biomedical signal processing, physiological monitoring, and embedded computing architectures. More specifically, the present invention relates to a method and system for the continuous, non-invasive estimation of mean arterial pressure (MAP) from a single-lead electrocardiogram (ECG) signal through multi-domain morphological analysis and deterministic classification suitable for real-time hardware and/or processor implementations.

Mean arterial pressure (MAP) is an essential physiological parameter reflecting the average arterial pressure during a complete cardiac cycle. Accurate and continuous MAP monitoring is critical for the early detection and management of hypertension and various cardiovascular risks. Traditional non-invasive blood pressure (NIBP) measurement typically utilizes cuff-based oscillometric devices. While generally accurate for spot-checks, such measurements are inherently intermittent, require active user participation, and cause discomfort during frequent inflation, making them unsuitable for continuous or autonomous monitoring.

To address these limitations, several “cuffless” approaches have been developed. These often rely on the fusion of multiple sensors, most commonly combining photoplethysmography (PPG) with ECG to compute pulse transit time (PTT) or pulse arrival time (PAT). For example, U.S. Patent Application Publication No. 2017/0340219 (Apple Inc.) and U.S. Pat. No. 12,239,424 (Fitbit Inc.) describe systems that use timing-based metrics as proxies to infer blood pressure. However, these multi-modal approaches suffer from technical drawbacks, including high sensitivity to motion artifacts and variability in PPG signal quality. Furthermore, as noted in the 2006 study by Payne et al., PTT measured solely from ECG markers can be an unreliable indicator of beat-to-beat blood pressure due to the decoupling of electrical and mechanical cardiac events.

Existing literature, such as U.S. Pat. No. 10,863,907 (CSEM) and U.S. Patent Application Publication No. 2013/0296723 (Samsung), discloses frameworks that depend on multimodal sensing or additional physiological inputs, such as electrical impedance tomography to achieve precision. While some machine learning-based attempts, such as those discussed in U.S. Patent Application Publication No. 2018/0116600, have sought to minimize inputs, they typically fail to establish a robust model based solely on ECG morphology and still require initial subject-specific calibration against a traditional cuff device to maintain accuracy.

Consequently, there remains a significant need for a fully non-invasive and continuous MAP estimation technique that: (i) relies strictly on a single ECG lead; (ii) eliminates the need for PPG and associated PTT/PAT derivations; (iii) operates without subject-specific calibration; and (iv) is executable with deterministic latency in low-power embedded hardware suitable for wearable “brain-heart axis” monitoring applications.

### BRIEF SUMMARY OF THE INVENTION

The present invention provides a method and system for the continuous, non-invasive, and calibration-free estimation of mean arterial pressure (MAP) utilizing a single-lead electrocardiogram (ECG) signal. The invention overcomes the limitations of traditional cuffless monitoring, such as dependence on multi-sensor fusion, pulse transit time (PTT) derivations, and frequent subject-specific recalibration, by deriving MAP directly from the multi-domain morphological characteristics of the ECG waveform itself.

In one embodiment, a digitized ECG signal is acquired at a fixed sampling rate and resolution. The signal is preprocessed through a cascaded filtering stage to mitigate baseline wander and powerline interference. Following preprocessing, the system identifies a plurality of fiducial points and landmarks corresponding to the P, Q, R, S, and T waves of the cardiac cycle.

The signal is segmented into overlapping analysis windows, each comprising two consecutive cardiac cycles with a one-cycle overlap. This windowing strategy enhances robustness against beat-to-beat variability and transient noise by ensuring every cycle is evaluated within a contextual temporal frame.

For each window, a Fiducial Mixture Model (FMM) Optimizer refines cardiac landmarks into twenty-one morphological parameters (e.g., amplitude, location, asymmetry, and sharpness) across the P, Q, R, S, and T waves. These morphological parameters are combined with nine temporal-dynamic features, such as segment inclinations and timings, to form a unified thirty-element feature vector.

A deterministic decision-tree classifier, implemented in specialized hardware such as an FPGA or ASIC, processes this vector against pre-quantized thresholds to output a MAP estimate via an AXI-compatible interface.

### BRIEF DESCRIPTION OF THE DRAWINGS

FIG. 1 is a high-level flowchart illustrating the sequential stages of the mean arterial pressure (MAP) estimation pipeline.

FIG. 2 is a schematic diagram illustrating the temporal segmentation of an ECG signal into overlapping sliding windows.

FIG. 3 is a block diagram illustrating the multi-domain architecture of the feature extraction module.

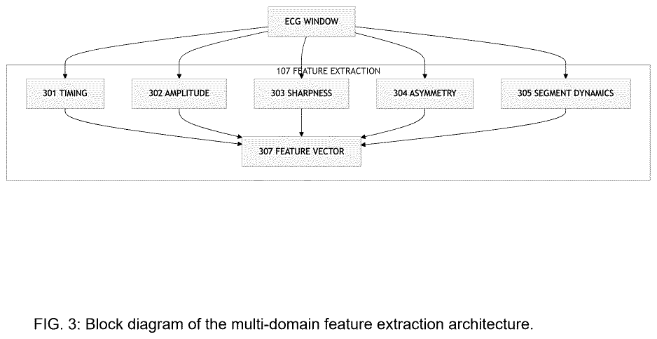

FIG. 4 is a flowchart illustrating the iterative optimization process within the Fiducial Mixture Model (FMM) engine.

FIG. 5 is a block diagram illustrating the deterministic decision tree classifier and its threshold evaluation logic.

FIG. 6 is a system architecture diagram illustrating the end-to-end hardware data path from signal input to AXI-compatible output.

FIG. 7 is a block-level schematic illustrating the minimal hardware implementation, including parameter-specific register banks and specialized arithmetic units.

### DETAILED DESCRIPTION OF THE INVENTION

The present invention is directed to a method and system for the continuous, non-invasive estimation of mean arterial pressure (MAP) from a single-lead ECG signal utilizing multi-domain morphological analysis and a deterministic classifier.

### I. Signal Acquisition and Preprocessing

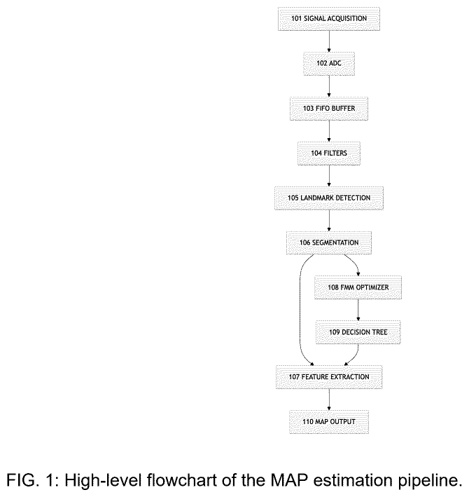

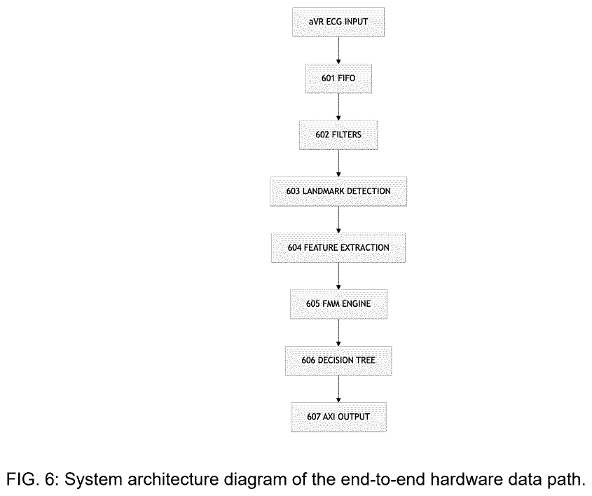

Referring to FIG. 1 and FIG. 6, the system receives a digitized ECG signal (such as an aVR ECG input) at a Signal Acquisition stage (101). While aVR is used in the preferred embodiment, the system is adaptable to any single-lead configuration with detectable P-T morphologies. To satisfy the requirements of low-power wearable hardware while preserving morphological integrity, the signal is sampled at a rate of at least 125 Hz.

A Filtering stage (104/602) performs noise mitigation. In a preferred embodiment, this involves a cascaded pipeline comprising:
A high-pass filter with a cut-off frequency of approximately 0.5 Hz to remove baseline wander.
A low-pass filter with a cut-off frequency between 40 Hz and 100 Hz to eliminate electromyographic (EMG) interference.
A notch filter tuned to 50 Hz or 60 Hz to mitigate powerline interference.

### II. Landmark Detection and Segmentation

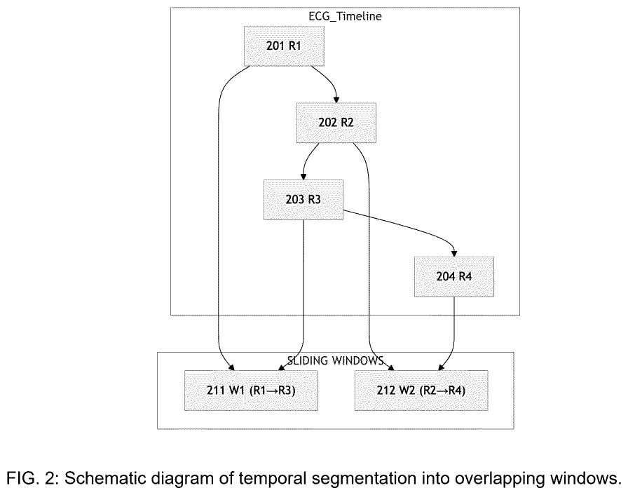

A Landmark Detection module (105/603) identifies primary fiducial points, specifically the R-peaks. As shown in FIG. 2, the signal is segmented into Sliding Windows (211, 212). Each window comprises two consecutive cardiac cycles (e.g., from 201 R1 to 203 R3) with a one-cycle overlap. This ensures that the TP-segment can be calculated by referencing the onset of the P-wave in the subsequent heartbeat.

### III. Fiducial Mixture Model (FMM) Optimizer

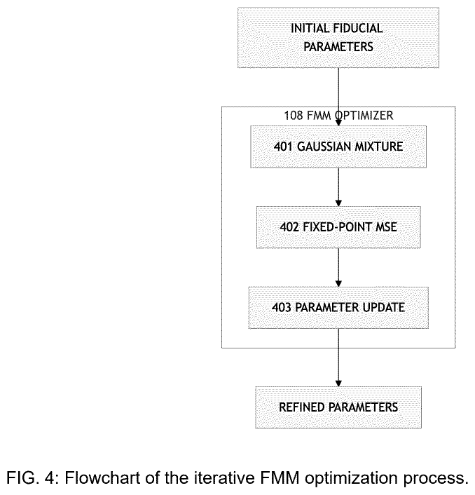

As illustrated in FIG. 4, the FMM Optimizer (108/605) refines parameters through a deterministic process. The system models the ECG waveform as a baseline offset M plus a summation of five wave components (P, Q, R, S, T). Each component i is defined by the FMM wave function:

$${W}_{i ( t )} = {A}_{i} \cdot cos ( {β}_{i} + 2 \cdot arctan ( {ω}_{i} \cdot tan ( \frac{t - {α}_{i}}{2} ) ) )$$

where A_{I} is amplitude, αᵢ is location, βᵢ is asymmetry, and ωᵢ is sharpness.

A Fixed-Point Mean Squared Error (MSE) (402) engine calculates the difference between the observed signal and the model, iteratively updating parameters to minimize the error:

$$MSE = \frac{1}{N} \sum {( {ECG}_{\{ obs \}} - ( M + \sum {W}_{i} ) )}^{2}$$

### IV. Multi-Domain Feature Extraction

The Feature Extraction engine (107/604) generates a thirty-element Feature Vector (307):
1. FMM Morphological Parameters (21): M and the four parameters (A, α, β, ω) for each of the five waves.
2. Timing Parameters (301): Including the PQ-interval, PQ-segment, PR-segment, ST-segment, and TP-segment.
3. Segment Dynamics (305): Specifically, the segment increments (inclinations) for the PQ, PR, ST, and TP segments. For example, the ST-increment is calculated as the voltage difference between the T-onset and the S-peak.

### V. Deterministic MAP Classification and Feature Importance

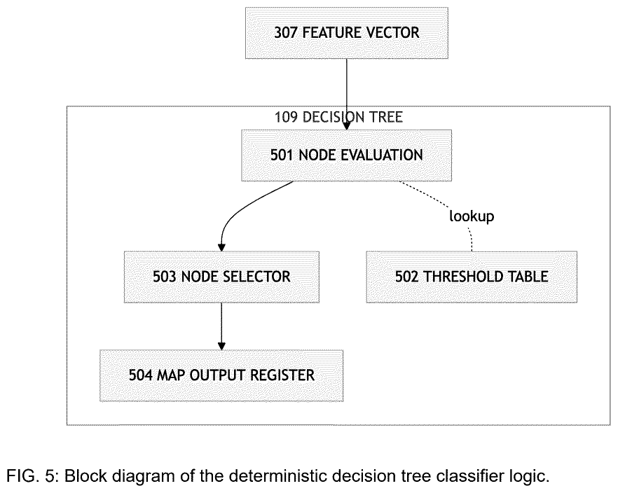

The feature vector (307) is passed to a Decision Tree Classifier (109/606). As shown in FIG. 5, the classifier performs Node Evaluation (501) using pre-quantized thresholds stored in a Threshold Table (502).

The hierarchy of the decision tree is informed by a technical analysis of feature importance, specifically measured by average gain. In the preferred embodiment, the system prioritizes the Location of the Q-wave (alpha_Q) and the Location of the T-wave (alpha_T), which demonstrate the highest correlation with arterial pressure fluctuations.

Furthermore, dynamic parameters such as the ST-segment inclination (st_segment_inc) and the PQ-interval (pq_interval) are utilized at high-level nodes of the tree to ensure the model captures both amplitude-based and timing-based morphological shifts. This structured prioritization allows the Node Selector (503) to traverse the tree with minimal logic operations, ensuring that a final estimate is stored in the MAP Output Register (504) and delivered via an AXI Output (607) with deterministic, low-latency performance suitable for real-time monitoring.

### VI. Hardware Minimal Implementation and Register-Level Architecture

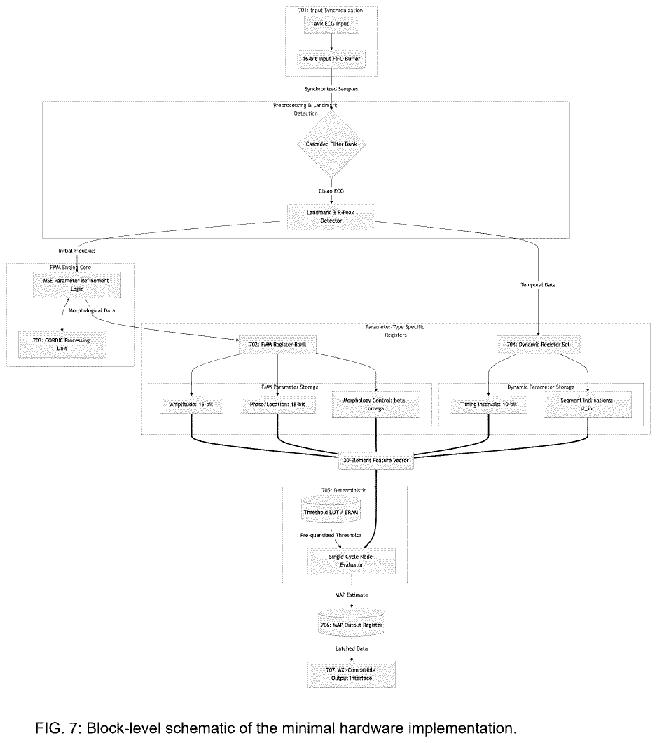

Referring to FIG. 7, the system is realized through a dedicated hardware data path optimized for minimal area and deterministic execution. The architecture transitions from the high-level functional blocks shown in FIG. 6 into a physical register-transfer level (RTL) structure where each processing stage is decoupled by parameter-type specific registers to ensure signal stability and predictable timing.

### A. Input Synchronization and Buffering (701)

The hardware data path initiates with a 16-bit Input FIFO Buffer (701) that functions as a synchronization bridge. This component effectively decouples the low-frequency sampling clock (e.g., 125 Hz) from the internal high-speed system clock of the FPGA or ASIC. This decoupling prevents sample loss and ensures that the downstream processing stages receive stable, jitter-free data windows for analysis.

### B. FMM Register Bank (702) and CORDIC Unit (703)

The FMM Register Bank (702) provides dedicated physical storage for the twenty-one morphological parameters generated by the FMM Engine. To optimize hardware area, the registers are partitioned as follows:
Amplitude Registers (A, M): Six registers utilizing 16-bit fixed-point precision to store wave peaks and baseline offsets.
Phase/Location Registers (α): Five 18-bit registers configured to store the high-precision temporal position of each wave relative to the R-peak.
Morphology Control: Registers specifically sized to store the asymmetry and sharpness parameters (β, ω) used during FMM wave synthesis.

To maintain a minimal hardware footprint, a CORDIC (Coordinate Rotation Digital Computer) unit (703) is integrated into the FMM core. This unit calculates the complex trigonometric components of the wave function using a hardware-efficient shift-and-add algorithm. This eliminates the need for resource-heavy floating-point multipliers, making the design suitable for low-power wearable ASICs.

### C. Dynamic Register Set (704) and Bit-Width Justification

The Dynamic Register Set (704) manages the nine features related to waveform timing and segments:
Timing Interval Registers: Utilizing a 10-bit width, these registers store integer sample counts for segments like the PQ and TP intervals. This width provides sufficient range (up to 1023 samples) to handle low heart rates at a 125 Hz sampling frequency while allowing the hardware to perform deterministic inclination scaling via bit-shifts.
Dynamic Inclination Registers: These store calculated voltage-shift values between fiducial points, representing the morphological rate of change.

### D. Deterministic Classifier (705) and AXI Output (707)

The Deterministic Classifier (705) is implemented as a combinational logic network that evaluates the 30-element Feature Vector in parallel. It retrieves pre-quantized thresholds from a Threshold LUT (stored in BRAM) and utilizes a Single-Cycle Node Evaluator to traverse the decision tree logic. By synchronizing this evaluation with the fixed-clock frequency of the system (e.g., 100 MHz), the architecture completes the traversal within a fixed number of clock cycles, thereby establishing a deterministic latency (e.g., 10 ns) and ensuring zero timing variance in the final estimate regardless of data complexity or system load.

The final MAP estimate is stored in the MAP Output Register (706), which latches the data only upon a completed valid cycle. The AXI-Compatible Output Interface (707) then formats this data into a standardized protocol, featuring Data, Valid, and Ready handshake signals, allowing the core to be integrated as a plug-and-play accelerator within larger medical monitoring systems.

1. A system for the continuous, non-invasive estimation of mean arterial pressure (MAP) from a single-lead ECG signal, the system comprising:
a signal acquisition module (701) configured to receive a digitized ECG signal;
a hardware-accelerated fiducial mixture model (FMM) engine (605) configured to model the ECG signal as a summation of wave components utilizing a CORDIC processing unit (703);
a parameter-specific register bank configured to store a thirty-element feature vector (307) derived from the FMM engine, the register bank comprising a plurality of 18-bit phase registers (702) and 10-bit timing interval registers (704); and
a deterministic decision-tree classifier (705) comprising a single-cycle node evaluator configured to process the thirty-element feature vector against a threshold lookup table to generate a MAP estimate with deterministic latency.

2. The system of claim 1, wherein the 18-bit phase registers (702) are configured to store temporal positions of P, Q, R, S, and T wave components with sub-sample resolution relative to a detected R-peak.

3. The system of claim 1, wherein the CORDIC processing unit (703) is configured to calculate trigonometric components of an FMM wave function utilizing a shift-and-add algorithm to eliminate floating-point multiplication.

4. The system of claim 1, wherein the 10-bit timing interval registers (704) are sized to accommodate integer sample counts for heart rates down to 40 beats per minute at a sampling rate of 125 Hz.

5. The system of claim 1, wherein the deterministic decision-tree classifier (705) is synchronized to a fixed-clock frequency of the system to establish a fixed physical latency independent of signal complexity.

6. The system of claim 1, wherein the deterministic decision-tree classifier (705) prioritizes a location parameter of a Q-wave and a location parameter of a T-wave as primary decision nodes within a classification hierarchy.

7. The system of claim 1, further comprising an AXI-compatible output interface (707) configured to present the MAP estimate as latched data to an external system-on-chip via a standardized handshake protocol.

8. A method for the continuous estimation of mean arterial pressure (MAP) from a digitized ECG signal, the method comprising:
preprocessing the ECG signal through a cascaded filtering stage to mitigate baseline wander and interference;
segmenting the ECG signal into overlapping analysis windows comprising at least two consecutive cardiac cycles;
refining cardiac landmarks into twenty-one morphological parameters using a fixed-point mean squared error minimization logic;
latching the twenty-one morphological parameters and nine temporal-dynamic parameters into a hardware register bank to form a thirty-element feature vector; and
traversing a hardware-implemented decision tree in a fixed number of clock cycles to produce a MAP value.

9. The method of claim 8, wherein segmenting the ECG signal utilizes a one-cycle overlap between consecutive windows to derive a TP-segment by referencing an onset of a P-wave in a subsequent heartbeat.

10. The system of claim 1, wherein the signal acquisition module, FMM engine, and deterministic classifier are integrated into a single wearable low-power ASIC for continuous cardiovascular monitoring.

11. The system of claim 1, wherein the FMM engine is configured to simultaneously refine amplitude (A), location (α), asymmetry β and sharpness (ω) for each of the five wave components (P, Q, R, S, T).

12. The system of claim 1, wherein the feature vector further comprises segment inclinations for the PQ, PR, ST, and TP segments calculated as a rate of voltage change between refined fiducial points.

13. The system of claim 1, wherein the signal acquisition module (701) includes a synchronization bridge configured to decouple the digitized ECG signal from the internal system clock of the FPGA or ASIC to prevent sample loss.

14. The method of claim 8, wherein the production of the MAP value is calibration-free and independent of subject-specific biometric inputs.
